(19) United States
(12) Patent Application Publication
(10) Pub. No.: US 2015/0289850 A1
(43) Pub. Date: Oct. 15, 2015

(54) Heat Dissipation Assemblies

(71) Applicant: Tyco Electronics Corporation, Berwyn, PA (US)

(72) Inventors: Kevin T. Lewis, Portland, OR (US); Jason M. Woods, Portland, OR (US); Dennis W. Dimick, Portland, OR (US)

(73) Assignee: Tyco Electronics Corporation, Berwyn, PA (US)

(21) Appl. No.: 14/253,162

(22) Filed: Apr. 15, 2014

(51) Int. Cl. A61B 8/00; F28D 15/00; F28F 13/00

(52) U.S. Cl. CPC A61B 8/546; F28F 13/00; F28D 15/00; A61B 8/4444; A61B 8/4483; A61B 8/4281; F28F 2013/006

(57) ABSTRACT

A coupling for interfacing a heat source to a heat sink for dissipating heat from the heat source includes a first portion and a second portion. The first portion defines a surface that defines a plurality of spaced apart voids that extend into the first portion. The second portion has an outside surface that complements the surface of the first portion. The second portion is capable of repeated mate and de-mate cycles from the first portion. A gel is disposed on the interior surface of the cavity. In operation, insertion of the second portion within the cavity causes a portion of the gel to displace into openings at first ends of the voids. The gel returns to an original shape when the second portion is removed from the cavity.

### FIELD

This application generally relates to heat dissipation for an electronic circuit. More, specifically, the application relates to various assemblies for dissipating heat from electronic components.

### BACKGROUND

Heat dissipation is an important consideration of many electrical systems. A heat sink, such as a block of metal with cooling fins may be pressed against a component to allow heat generated from the component to dissipate. Thermal grease may be applied between the component and the heat sink to improve the thermal conductivity between the component and the heat sink. The thermal grease operates by filling in micro voids on the mating surfaces. This in turn increases the effective thermal conductivity of the contact area between the surfaces, which lowers the thermal resistance.

After the thermal grease is applied, the heat sink is secured to the component via clips or screws. If subsequent removal and reattachment of the heat sink is required, an additional application of thermal grease may be required as the thermal grease tends to become unevenly distributed across the surfaces of the parts.

An ultra sound system is but one example of an electrical system in which heat dissipation is an important consideration. An ultra sound system typically includes an ultra sound probe with an embedded transducer. The transducer generates ultra sonic waves, which are read by the system to generate an image. The transducer is typically an electro-mechanical device, such as a piezoelectric material, that vibrates and, therefore, generates heat. It may also include electrical signal processing devices, such as an integrated circuit, that processes data and, therefore, generates heat.

To prevent patient discomfort, the system may include a cooling system for cooling the probe. For example, a large area of the probe may be dedicated to heat sinking of the transducer. Reserving such an area increases the size of the probe, which may lead to more hand discomfort for the operator.

### BRIEF DESCRIPTION

In a first aspect, a coupling for interfacing a heat source to a heat sink for dissipating heat from the heat source includes a first portion and a second portion. The first portion defines a surface that defines a plurality of spaced apart voids that extend into the first portion. The second portion has an outside surface that complements the surface of the first portion. The second portion is capable of repeated mate and de-mate cycles from the first portion. A gel is disposed on the interior surface of the cavity. In operation, insertion of the second portion within the cavity causes a portion of the gel to displace into openings at first ends of the voids. The gel returns to an original shape when the second portion is removed from the cavity.

In a second aspect, a heat absorption assembly for cooling a component includes a cooling module, a gel layer, and a component coupler. The cooling module includes a housing with a surface configured to contact a surface of the component. A phase change material (PCM) material is disposed within the housing. The phase change material transitions between liquid and solid states at the same temperature. The component coupler is disposed on a component separated from the cooling module. The component coupler defines a plurality of cavities. The gel layer is disposed on an outside surface of the component coupler. When the cooling module is pressed against the component coupler, the gel layer between the two parts at least partially displaces within the cavities of the component coupler. When the cooling module is removed from the component, the gel layer returns to its original shape, thus facilitating repeated attachment and removal of the cooling module to the component.

In a third aspect, an ultrasound probe includes a probe housing, a transducer module is disposed within the probe housing, a plurality of electronic components used to activate the transducer module, and a heat absorption assembly. The transducer module includes a component that facilitates the dissipation of heat. The heat absorption module is configured to draw heat away from the transducer module and the electronics. The heat absorption assembly includes a housing and a phase change material (PCM) material disposed within the housing. The phase change material transitions between liquid and solid states at the same temperature.

In a fourth aspect, a recharging station includes a housing that defines one or more cavities configured to receive one or more cooling modules, and a cooling device with a cooling capability disposed within the housing. When one or more cooling modules are inserted into the one or more cavities, the cooling device cools the one or more cooling modules. The cooling capability of the cooling device may be selectively activated.

In a fifth aspect, a re-cooling station includes a housing that defines one or more cavities configured to receive one or more cooling modules. A cooling device with a cooling capability is disposed within the housing. When the cooling modules are inserted into the one or more cavities, the cooling device is configured to cool the cooling modules. The cooling capability of the cooling device may be selectively activated.

### BRIEF DESCRIPTION OF THE DRAWINGS

The accompanying drawings are included to provide a further understanding of the claims, are incorporated in, and constitute a part of this specification. The detailed description and illustrated embodiments described serve to explain the principles defined by the claims.

FIGS. 1A and 1B illustrate a first exemplary coupling for interfacing a heat source to a heat sink for dissipating heat from the heat source;

FIGS. 1C and 1D illustrate a second exemplary coupling for interfacing a heat source to a heat sink for dissipating heat from the heat source;

FIG. 2 illustrates an exemplary cross-section of a portion of the coupling of FIGS. 1A and 1B taken along cross-section A-A′;

FIGS. 3A and 3B illustrate exemplary side and top views, respectively, of a device for cooling a component;

FIGS. 4A-F illustrate exemplary views of a heat absorption assembly for cooling a component;

FIG. 5 illustrates an exemplary ultrasound probe adapted to utilize a heat absorption assembly; and

FIG. 6 illustrates an exemplary re-cooling station for recharging a cooling component.

### DETAILED DESCRIPTION

A variety of heat dissipation embodiments are disclosed below. Some of the embodiments overcome problems discussed above by providing a heat conducting gel layer between a heat source and a heat sink. The gel layer is self-healing and the assemblies include gaps or cavities that allow the gel layer to displace when a heat source component and heat sink component are coupled/squeezed together. The gel layer facilitates repeated coupling and de-coupling of the heat source to the heat sink.

Other heat dissipation embodiments include a phase change material (PCM) device that maintains the temperature of a component below a threshold temperature until the heat capacity of the PCM is reached. These embodiments combine the features of the PCM device with the gel layer discussed above to provide a heat dissipation assembly that can be easily attached to and removed from a component to be cooled without the issues discussed above with respect to thermal grease.

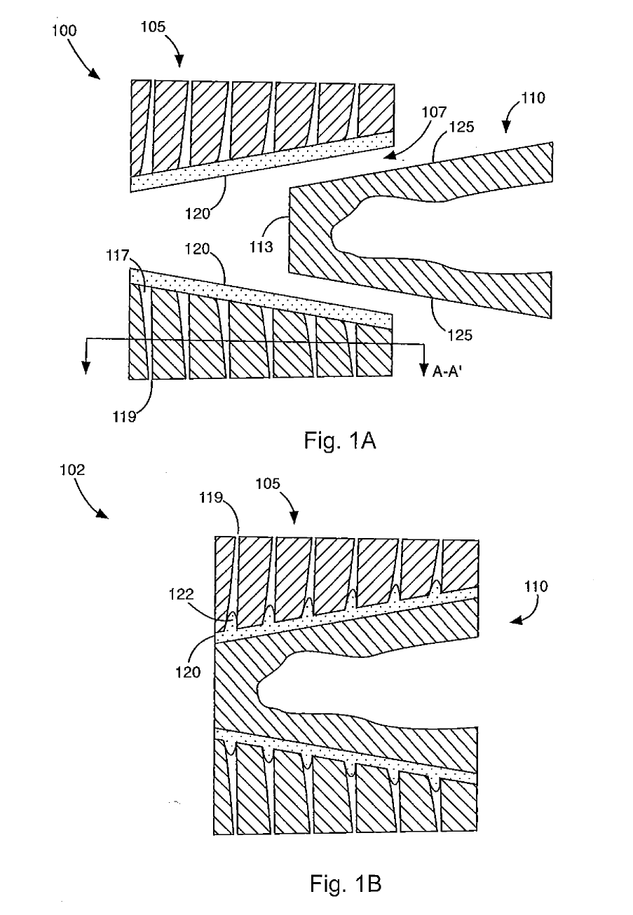

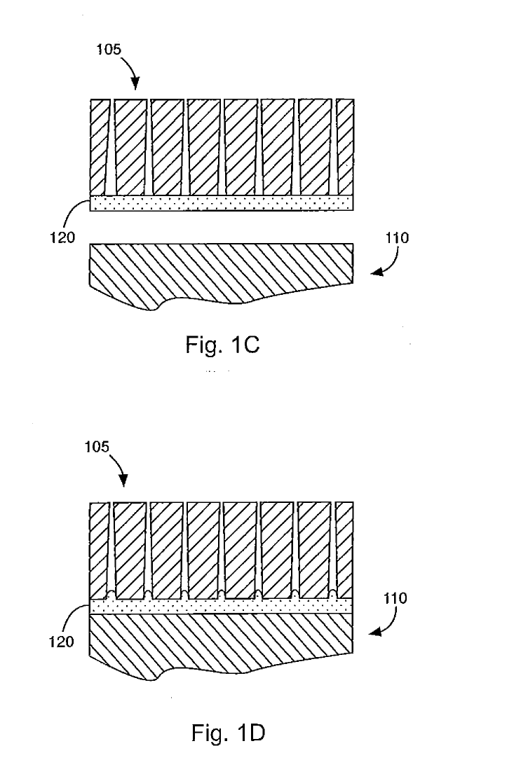

FIGS. 1A and 1B illustrate a coupling 100 for interfacing a heat source to a heat sink for dissipating heat from the heat source. The coupling 100 includes a first portion 105 that defines a cavity 107, and a second portion 110 that is insertable into the cavity. The cavity 107 may taper gradually with distance away from the opening into which the second portion 110 is inserted. The second portion 110 has an outside surface 125 that complements the interior surface geometry of the cavity 107. Other configurations are possible.

In some implementations, electrical signals and the like may be coupled via the coupling 100. For example, the forward surface 113 of the second portion 110 and the inner most section of the first portion 105 may include complementary male and female electrical contacts.

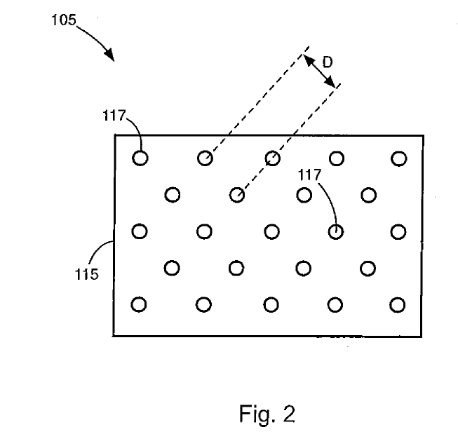

The interior surface of the cavity 107 defines a plurality of spaced apart hollow voids 117 that extend into the first portion 105. The voids 117 may be arranged in a variety of different configurations, such as the configuration illustrated in FIG. 2. In an exemplary configuration, the distance, D, between adjacent voids 117 may be about 0.300 inches. The diameter of each channel may be about 0.100 inches. The interior shape of the voids 117 may be cylindrical, frustoconical, channular or a different shape.

The first portion 105 of the coupling 100 may form part of or be connected to a heat sink, such as a block of metal with cooling fins, a fan, etc., while the second portion 110 may form part of or be connected to equipment that is generating heat. The opposite configuration is also possible. In an exemplary implementation, the first portion 105 may form part of a heat sinking section of a backplane of an equipment rack. The second portion 110 may form part of a module that is inserted into the equipment rack. The taper of the cavity 107 facilitates alignment of the second portion 110 within the first portion 105 and allows for easy insertion of the second portion 110 from within the cavity 107 (FIG. 1B) and removal thereof (FIG. 1A). The taper of the cavity 107 also allows one to generate a compressive force on the gel without any additional mechanical movement aside from the insertion of the second portion 110 into the first portion 105. Other configurations, in which a taper is not used, are possible. For example, as illustrated in FIGS. 1C and 1D, a parallel insertion of the second portion 110 into the first portion 105 where in the gel compression is generated by a cam feature (not shown).

A gel 120 is disposed on the interior surface of the cavity 105. The gel 120 may be a thermally conductive gel or other thermal interface material. When the second portion 110 is inserted into the cavity 107, a portion 122 of the gel 120 displaces into openings at first ends of the hollow voids 117. Displacement of the portions 122 of the gel 120 into the voids 117 increases the contact area between the gel 120 and the first portion 105. It also allows the thickness of the gel 120 to decrease. For example, the thickness of the gel may decrease to about 0.76 mm (0.030 inch). The decrease in the thickness of the gel 120 results in the inside surface of the cavity 107 of the first portion 105 and the outside surface 125 of the second portion 110 coming closer together. The decrease in thickness of the gel 120 decreases the thermal resistance of the gel 120. For example, the thermal resistance may decrease to about 10 cm²-K/W.

In some implementations, each hollow void 117 may define an opening at a second end 119 that facilitates equalization of pressure within the channel 117 to atmospheric pressure when the gel 120 is displaced into the void 117. This in turn reduces the resistance exhibited when inserting the second portion 110 into the cavity 107 by allowing the gel 120 to more easily displace. In some implementations, the hollow voids 117 may not define an opening on a second end. This allows for the buildup of pressure behind the gel 120 during displacement, which can aid in the ejection of the gel 120 from the void upon decoupling of the first portion 105 and the second portion 110.

The gel 120 may be a so-called self-healing gel, which is a gel that returns to its original unstressed shape when pressure is removed from the gel 120. In this case, the gel 120 will return to its original shape or very close to its original shape when the second portion 110 is removed from the cavity 107. For example, the gel 120 may be a silicone gel such as gels sold as Dow Corning® GT gels or a different gel material that exhibits a characteristic predisposition to return to its original shape after deformation or separation. The distance, D, between the voids may be adjusted to provide a desired amount of resistance to insertion of the first portion 105 within the second portion 110. For example, the number of voids may be increased and the spacing between channels may be decreased to provide more channels for displacement, which reduces the insertion resistance.

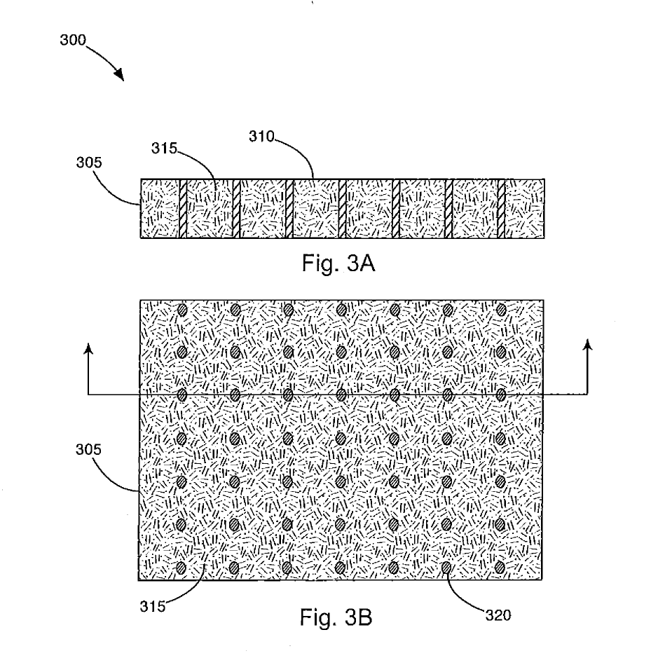

FIGS. 3A and 3B illustrate exemplary side and top views, respectively, of a device 300 for cooling a component. The device 300 may be utilized in combination with the coupling 100, described above. The device 300 includes a generally hollow housing 305. In the exemplary views, the device 300 includes a generally planar surface that in operation contacts a corresponding planar surface of the component. However, the surface may have a different shape, such as a concave, convex, or a different shape that complements the surface of the component. Optimal cooling capability is achieved when the surface of the device 300 complements the corresponding surface of the component.

A phase change material (PCM) material 315 is disposed within the housing 305. In some implementations, the PCM 315 is a material that transitions from a solid state to a liquid state and from a liquid state to a solid state at a same temperature. For example, eicosane or a different organic PCM may be selected. Such PCMs transition between a solid state and a liquid state at a same temperature. Eicosane, as an exemplary PCM, transitions at about 37° C.

To provide for more uniform heat transfer and a uniform melt and freezing condition throughout the device 300, the internal structure of the housing 305 may include a network of conduits 320 that transfer heat throughout the PCM 315 and introduce nucleation sites for the PCM phase change along the surfaces of the network of conduits 320. For example, a group of metal conduits 320 with rough surfaces may extend from a bottom surface of the housing 310 to a top surface of the housing 310, as illustrated in FIG. 3A. Alternative implementations may include providing a honeycomb network of conduits, providing a mesh screen, or extending the conduits from side-to-side rather than or in addition to top-to-bottom. Other implementations are possible. The PCM 315 may be disposed outside of or inside of the conduits.

In some implementations, thermally conductive additives may be added to the PCM 315 to enhance heat transfer and melting/freezing characteristics throughout the PCM 315. For example, aluminum beads or other additives with similar properties may be added. The additives may result in the creation of multiple nucleation sites, which creates multiple locations within the PCM 315 for solidification and liquidation of the PCM to occur. The nucleation sites help to ensure that the PCM 315 will have a repeatable and consistent phase change process at a same temperature, which will allow for improved performance and reliability of the device. The abundance of nucleation sites will also cause air entrapment as little bubbles throughout the PCM when it freezes, instead of creating a single large air volume. This in turn continues to increase the reliability and repeatability of the thermal performance of the PCM pack by allowing the heat to easily access and melt the PCM in any geometric orientation of the probe.

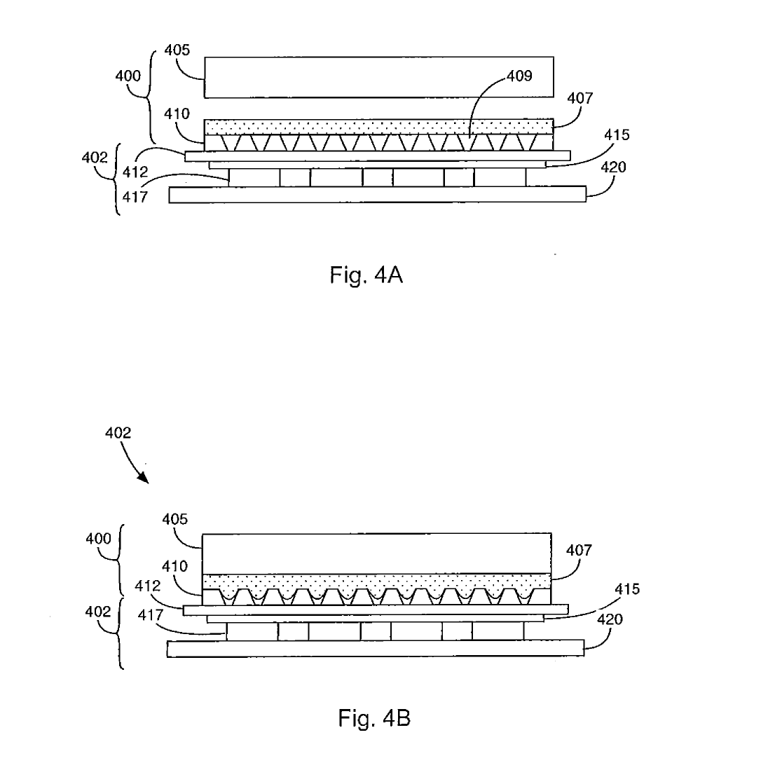

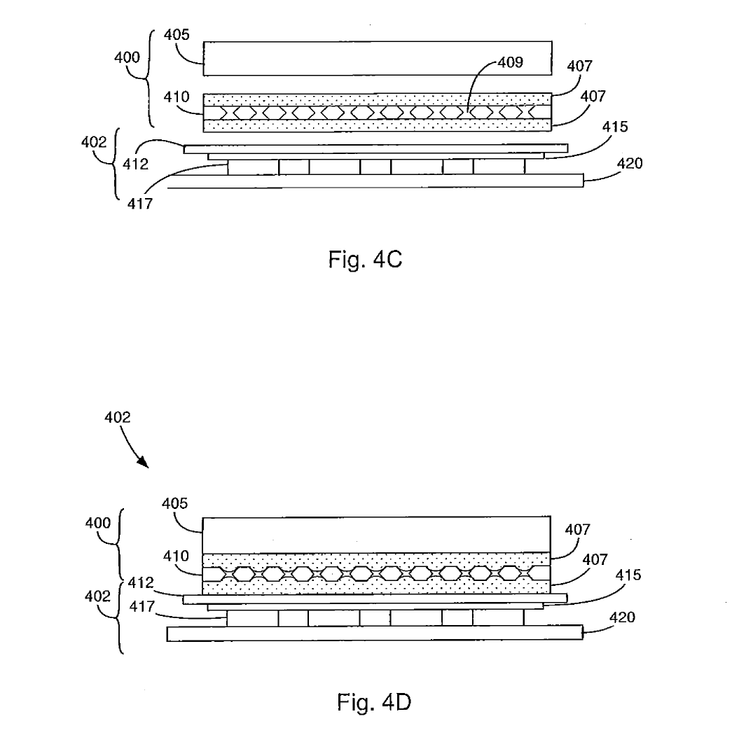

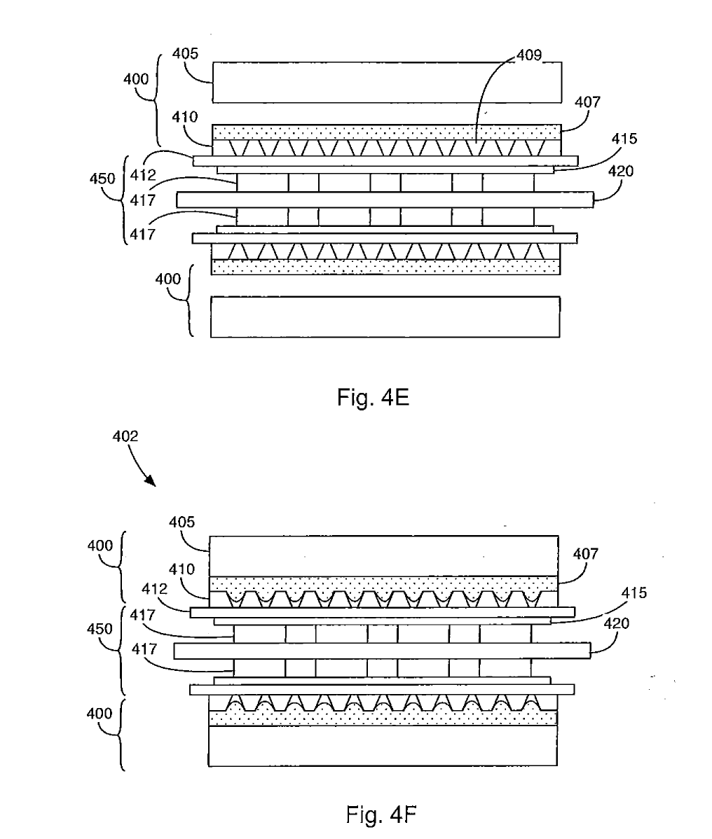

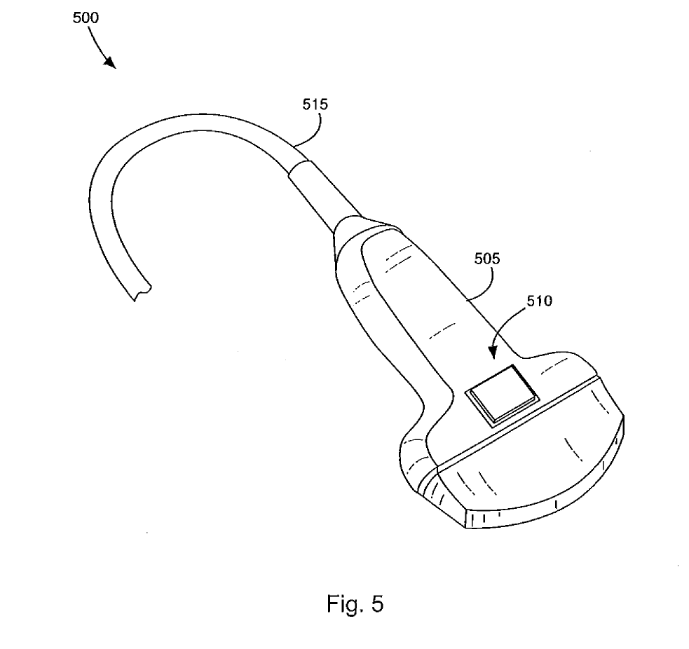

FIGS. 4A and 4B illustrate exemplary views of a heat absorption assembly 400 for cooling a component. The heat absorption assembly 400 includes a cooling module 405 that may correspond to the device 300 for cooling a component, described above. For example, the cooling module 405 may include a housing and PCM disposed within the housing. The housing may have a generally planar lower surface. The PCM may transition between liquid and solid states at a same temperature.

A gel layer 407 is disposed on a surface of a component coupler 410. The gel layer 407 may comprise a gel such as the gel 120 described above. For example, the gel layer 407 may be thermally conductive. The gel layer 407 may be a self-healing gel so that it returns to its original shape when not stressed. The thickness of the gel layer 407 when not in stress may be about 0.050″.

A component coupler 410 is disposed on a heat source or heat sink component. The component coupler 410 defines a plurality of cavities 409. The cavities 409 may be tapered with the wider side being on the side of the component coupler 410 closer to the gel layer 407. (I.e., the side of the component coupler disposed towards the gel layer 407.) In some implementations, the cavities 409 may extend from a top side of the component coupler 410 to a bottom side of the component coupler 410.

The heat absorption assembly 400 may be utilized to cool components that are part of a component assembly 402. The component assembly 402 may include a group of heat generating components 417 arranged on a printed circuit board 420. A thermal interface material (TIM) 415 may be applied on a top surface of the components 417. For example, heat grease may be applied. A heat plate 412 made of a material with low thermal resistance, such as copper, graphite, or aluminum is arranged over the TIM 415 and the components 417. The heat plate acts to spread the heat in plane as well as conduct the heat through the material.

The heat absorption assembly 400 (hereinafter HAA 400) is arranged above the heat plate 412 and a force is applied to press the HAA 400 against the heat plate 412. When the HAA 400 is pressed against the heat plate 412, the gel layer 407 at least partially displaces within the cavities 409 of the component coupler 410, as illustrated in FIG. 4B. Displacement of the gel layer 407 into the cavities 409 increases the contact area between the gel layer 407 and the component coupler 410. The gel layer 407 is also thinned. For example, the thickness of the gel layer 407 may change to about 0.50mm±0.25mm (0.020±0.010 inch) when the heat absorption assembly 400 is pressed against the heat plate 412. The increased contact area and decrease in thickness decreases the thermal resistance of the gel layer to about 10 cm²-K/W.

When the PCM in the cooling module 405 has completely changed to a liquid state, the HAA 400 may be removed from the heat plate 412 and put aside to allow the PCM to cool. A second HAA 400 with a cooled PCM may be pressed against the component assembly 402. Between HAA 400 exchanges, the displaced gel layer 407 of the first mating assembly will generally return to its original shape. The first HAA 400 may be re-used after the PCM has cooled.

In this manner, a number of heat absorption assemblies may be interchanged repeatedly and rapidly to cool the component assembly for an extended period of time.

Other implementations are possible. For example, as illustrated in FIGS. 4C and 4D, gel layers 407 may be provided on both sides of the component coupler 410. As illustrated in FIGS. 4E and 4F, a pair of HAAs 400 may be utilized to cool a component assembly 450 that includes components 417 on both a top side and a bottom side of a PCA 420. In some implementations, a heat plate 412 or TIM 415 may not be necessary.

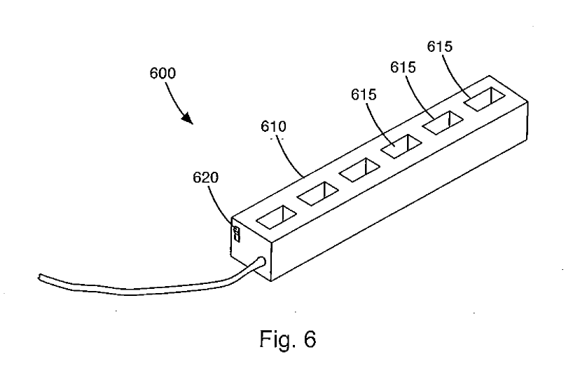

In some implementations, a re-cooling station 600 (FIG. 6) for more rapidly cooling the cooling modules 405 may be provided. Referring to FIG. 6, the re-cooling station 600 may include a housing 610 that defines one or more cavities 615 configured to receive one or more cooling modules 405. A cooling device (not shown) may be disposed within the housing for cooling the HAAs. The cooling device may, for example, correspond to one or more peltier devices, a compressor based cooling system, a large heat sink with or without a fan, etc. When the cooling modules 405 are inserted into the cavities 615, the cooling device cools the cooling modules 405. The cooling modules 405 may be cooled until they freeze. The re-cooling station 600 may draw power in operation, in which case a switch 620 may be provided to activate and deactivate the re-cooling station 600. The switch 620 may be on the side of the re-cooling station 600, or disposed within the cavities 615, such that insertion of a cooling module 405 automatically activates the re-cooling station 600.

One application where this arrangement is particularly useful is in ultra sound imaging. As noted above, ultrasound imagers typically include transducers, but may also include electronics, transceivers, and optical components, all of which generate heat. As illustrated in FIG. 5, an ultrasound probe 500 may be adapted to utilize a heat absorption assembly, such as the HAA described above. For example, an opening 510 may be provided in the probe housing to expose a heat generating surface of the probe. The HAA may then be pressed onto the transducer with enough pressure to cause the gel layer to displace the appropriate amount. The HAA may be secured in place via clips or other common locking mechanism (e.g. CAM lock, screws, interference fit, etc.). An electronic or visual indicator (not shown) may be provided to alert an operator when the heat capacity limit of the HAA has been reached. The operator may then remove the HAA, set it aside to cool, and then attach an already cooled HAA to the probe and continue working.

Cooling the probe 505 with an HAA rather than providing cooling lines and heat sinks allows for the construction of a smaller cable 515. The smaller cable 515 will result in less fatigue for the operator.

While various embodiments have been described, it will be apparent to those of ordinary skill in the art that many more embodiments and implementations are possible that are within the scope of the claims. Therefore, the embodiments described are only provided to aid in understanding the claims and do not limit the scope of the claims.

What is claimed is:

1. A coupling for interfacing a heat source to a heat sink for dissipating heat from the heat source comprising:
a first portion that defines a surface, wherein the surface defines a plurality of spaced apart voids that extend into the first portion;
a second portion with an outside surface that complements the surface of the first portion, the second portion being capable of repeated mate and de-mate cycles from the first portion;
a gel disposed on the surface of the first portion, wherein mating of the second portion with the first portion causes a portion of the gel to displace into openings at first ends of the voids, and wherein the gel returns to an original shape when the second portion is removed from the cavity.

2. The coupling according to claim 1, wherein the first portion forms part of the heat sink and the second portion forms part of the heat source.

3. The coupling according to claim 1, wherein the first portion forms part of the heat source and the second portion forms part of the heat sink.

4. The coupling according to claim 1, wherein each void defines an opening at a second end that facilitates equalization of pressure within the channel to atmospheric pressure when the gel is displaced within the channel.

5. The coupling according to claim 1, wherein each void is closed at a second end, wherein pressure within the void is increased when the gel is displaced into the void, and wherein the increased pressure decreases a force required to decouple the second portion from the first portion.

6. The coupling according to claim 1, wherein the gel is a self-healing gel selected from the group of gels consisting of: silicone gels and other gels exhibiting a similar characteristic predisposition to return to their original shape after deformation or separation.

7. The coupling according to claim 1, where the surface corresponds to an interior surface of a cavity.

8. A heat absorption assembly for cooling a component, the heat absorption assembly comprising:
a cooling module that includes:
a housing with a surface configured to contact a surface of the component; and
a phase change material (PCM) material disposed within the housing, wherein the phase change material transitions between liquid and solid states at a same temperature;
a component coupler disposed on a component separated from the cooling module, wherein the component coupler defines a plurality of cavities; and
a gel layer disposed on an outside surface of the component coupler wherein when the cooling module is pressed against the component coupler, the gel layer between the two parts at least partially displaces within the cavities of the component coupler, and wherein when the cooling module is removed from the component, the gel layer returns to an original shape, thus facilitating repeated attachment and removal of the cooling module to the component.

9. The heat absorption assembly according to claim 8, wherein an internal structure of the housing defines a network of conduits, wherein the phase change material is disposed outside of or inside of the conduits, and wherein the conduits facilitate heat transfer and provide nucleation sites throughout the phase change material.

10. The heat absorption assembly according to claim 8, wherein the phase change material comprises additives that facilitate the creation of a plurality of nucleation sites within the phase change material.

11. The heat absorption assembly according to claim 10, wherein the additives are selected from a group of additives consisting of: polymers, metals, ceramics, composites or mixtures thereof.

12. The heat absorption assembly according to claim 8, wherein the gel is a self-healing gel selected from the group of gels consisting of: silicone gels and other gels exhibiting a similar characteristic predisposition to return to their original shape after deformation or separation.

13. An ultrasound probe comprising: a probe housing;
a transducer module disposed within the probe housing, wherein the transducer module includes a component that facilitates the transfer of heat; and
a plurality of electronic components used to activate the transducer module; and
a heat absorption assembly configured to draw heat away from the transducer module and electronics, wherein the heat absorption assembly includes:
a housing; and
a phase change material (PCM) material disposed within the housing, wherein the phase change material transitions between liquid and solid states at a same temperature.

14. The ultrasound probe according to claim 13, wherein the heat absorption assembly is removable and further includes:
a component coupler with a first side disposed on a surface of the component of the transducer module or on a surface of the electronic components, wherein the component coupler defines a plurality of cavities; and
a gel layer disposed on one or more of an outside surface of the component coupler and an outside surface of the housing, wherein when the heat absorption assembly is pressed against the transducer module, the gel layer at least partially displaces within the cavities of the coupler, and wherein when the heat absorption assembly is removed from the ultrasound probe, the gel layer returns to an original shape, thus facilitating repeated attachment and removal of the heat absorption assembly to the transducer.

15. The ultrasound probe according to claim 14, wherein the gel is a self-healing gel selected from the group of gels consisting of: silicone gels and other gels exhibiting a similar characteristic predisposition to return to their original shape after deformation or separation.

16. The ultrasound probe according to claim 13, wherein an internal structure of the housing defines a network of conduits, wherein the phase change material is disposed outside of the conduits, and wherein the conduits facilitate heat transfer throughout the phase change material.

17. The ultrasound probe to claim 13, wherein the phase change material comprises additives configured to facilitate the creation of a plurality of nucleation sites within the phase change material.

18. The device according to claim 17, wherein the additives are selected from a group of additives consisting of: metals, ceramics, polymers, composites, or a mixture thereof.

19. The ultrasound probe according to claim 14, where the probe housing defines an opening that facilitates access to the heat absorption assembly, wherein the heat absorption assembly is configured to be selectively removed from the probe housing.

20. A re-cooling station comprising:
a housing that defines one or more cavities configured to receive one or more cooling modules; and
a cooling device with a cooling capability disposed within the housing, wherein when the one or more cooling modules are inserted into the one or more cavities, the cooling device is configured to cool the one or more cooling modules, and wherein the cooling capability of the cooling device may be selectively activated.

21. The re-cooling station according to claim 20, wherein each of the cooling modules comprises:
a housing with an exterior geometry that complements an interior geometry of one of the one or more cavities; and
a phase change material (PCM) disposed within the housing that melts and freezes at a same temperature, wherein when the housing is inserted into a cavity of the one or more cavities, the PCM is cooled and freezes when below the melt temperature.
